SEQUENCE LISTING (13 sequences)

SEQ ID NO: 1 - protein, 27 aa, Homo sapiens
GYVSGWGRNANFKFTDHLKYVMLPVAD

SEQ ID NO: 2 - protein, 388 aa, Homo sapiens
VDSGNDVTDIADDGCPKPPEIAHGYVEHSVRYQCKNYYKLRTEGDGVYTLNDKKQWINKAVGDKLPECEADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 3 - protein, 245 aa, Homo sapiens
ILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 4 - protein, 21 aa, Homo sapiens
LGGHLDAKGSFPWQAKMVSHH

SEQ ID NO: 5 - protein, 16 aa, Homo sapiens
LTTGATLINEQWLLTT

SEQ ID NO: 6 - protein, 25 aa, Homo sapiens
LYVGKKQLVEIEKVVLHPNYSQVDI

SEQ ID NO: 7 - protein, 29 aa, Homo sapiens
LIKLKQKVSVNERVMPICLPSKDYAEVGR

SEQ ID NO: 8 - protein, 20 aa, Homo sapiens
DQCIRHYEGSTVPEKKTPKS

SEQ ID NO: 9 - protein, 36 aa, Homo sapiens
VGVQPILNEHTFCAGMSKYQEDTCYGDAGSAFAVHD

SEQ ID NO: 10 - protein, 22 aa, Homo sapiens
EEDTWYATGILSFDKSCAVAEY

SEQ ID NO: 11 - protein, 19 aa, Homo sapiens
VYVKVTSIQDWVQKTIAEN

SEQ ID NO: 12 - protein, 15 aa, Homo sapiens
CYEGSTVPEKKTPKS

SEQ ID NO: 13 - protein, 15 aa, Homo sapiens
CGLIKLKQKVSVNER

(19) United States
(12) Patent Application Publication
(10) Pub. No.: US 2018/0344808 A1
(43) Pub. Date: Dec. 6, 2018

(54) HAPTOGLOBIN DERIVATIVE FOR TREATMENT OF SEPSIS AND ACETAMINOPHEN-INDUCED LIVER DAMAGE

(71) Applicant: THE FEINSTEIN INSTITUTE FOR MEDICAL RESEARCH, Manhasset, NY (US)

(72) Inventors: Kevin J. Tracey, Old Greenwich, CT (US); Huan Yang, Oakland Gardens, NY (US)

(73) Assignee: THE FEINSTEIN INSTITUTE FOR MEDICAL RESEARCH, Manhasset, NY (US)

(21) Appl. No.: 15/991,259

(22) Filed: May 29, 2018

Related U.S. Application Data
(60) Provisional application No. 62/513,557, filed on Jun. 1, 2017.

(51) Int. Cl. A61K 38/17; A61K 47/60; A61K 47/64; A61K 9/00; A61P 31/02

(52) U.S. Cl. CPC A61K 38/1709; A61K 47/60; A61P 31/02; A61K 9/0019; A61K 47/643

(57) ABSTRACT

Methods for treating sepsis of acetaminophen-induced liver damage in a subject sing a haptoglobin derivative are provided. Compositions containing a haptoglobin derivative for treating sepsis of acetaminophen-induced liver damage are provided.

### CROSS-REFERENCE TO RELATED APPLICATIONS

This application claims benefit of U.S. Provisional Application No. 61/513,557, filed Jun. 1, 2017, the contents of which are hereby incorporated by reference.

### GOVERNMENT SUPPORT CLAUSE

This invention was made with government support under grant number GM098446 awarded by the National Institutes of Health. The government has certain rights in the invention.

### BACKGROUND OF THE INVENTION

Throughout this application various publications are referred to. The disclosures of these publications, and of all patents, patent application publications and books referred to herein, are hereby incorporated by reference in their entirety into the subject application to more fully describe the art to which the subject invention pertains.

Sepsis is a common and difficult to treat pathology with a high mortality rate. There are more than 1 million cases of sepsis each year, according to the Centers for Disease Control and Prevention (CDC) and more than 258,000 resultant fatalities in the U.S.

The present invention addresses the need for improved methods for treating sepsis, and also for treating acetaminophen-induced liver damage.

### SUMMARY OF THE INVENTION

A method of treating sepsis in a subject is provided, comprising administering to the subject an amount of composition comprising a peptide having SEQ ID NO:1, but not comprising SEQ ID NO:2, effective to treat sepsis in a subject.

Also provided is a method of reducing the likelihood of mortality from sepsis in a subject having the sepsis, comprising administering to the subject an amount of composition comprising a peptide having SEQ ID NO:1, but not comprising SEQ ID NO:2, effective reduce the likelihood of mortality of a subject from sepsis.

Also provided is a method of inhibiting HMGB1-induced TNF release from a macrophage in a subject, comprising administering to the subject an amount of composition comprising a peptide having SEQ ID NO:1, but not comprising SEQ ID NO:2, effective to inhibit HMGB1-induced TNF release from a macrophage in a subject.

Also provided is a method of treating acetaminophen-induced liver damage in a subject, comprising administering to the subject an amount of composition comprising a peptide having SEQ ID NO:1, but not comprising SEQ ID NO:2, effective to treat acetaminophen-induced liver damage in a subject.

Also provided is a composition comprising a peptide having SEQ ID NO:1, but not comprising SEQ ID NO:2, wherein the peptide is fused to a molecule that increases plasma-half life of the peptide.

### BRIEF DESCRIPTION OF THE DRAWINGS

FIG. 1A-1B: 1A. Free hemoglobin is ubiquitous and enhances LPS-mediated inflammation. 1B. We initially developed a treatment method using haptoglobin-beads to remove free hemoglobin from septic rats. Unexpectedly, we pulled out HMGB1 using haptoglobin linked to sepharose beads in an extracorporeal circulatory device, besides hemoglobin.

FIG. 2A-2B: 2A. Human haptoglobin has three phenotypes and contains α and β subunits. 2B. Similar as full length haptoglobin (JCI Insight, 2016), haptoglobin β binds to HMGB1 with high affinity (Kd=29 nM) and in a concentration-dependent manner using surface plasmon resonance analysis. Data shown is representative of 3 separate experiments.

FIG. 3A-3B: HMGB1 stimulated TNF release in murine macrophage-like RAW 264.7 cells. 3A. The addition of haptoglobin produced a dose-dependent attenuation of the HMGB1-induced TNF release (up to 90% inhibition at 100 μg/ml). 3B. Haptoglobin β is as effective as haptoglobin in inhibiting HMGB1-induced TNF release. N=3-5 experiments. *: P<0.05 vs. HMGB1 alone.

FIG. 4A-4B: To elucidate the links between haptoglobin, HMGB1 and sepsis, we studied mice with Hp knockout (KO). 4A. Hp KO mice, following CLP, suffered a doubling of mortality compared to wild type mice. 4B. In Hp KO mice after CLP, serum HMGB1 levels were persistently higher for at least 21 days compared to wild type mice, suggesting HMGB1 may play a role in increased mortality in Hp KO mice. *:P<0.05 vs. WT.

FIG. 5A-5B: 5A Wild type mice subjected to cecal ligation and puncture (CLP) who received injections of haptoglobin β (200 μg/mouse injected IP) were twice as likely to survive as compared to vehicle-treated (control) group (*: P<0.05). N=16 mice/group. 5B. Haptoglobin KO mice were rescued from death by administration of haptoglobin (3 at doses as low as 100 μg per animal per day. Recombinant haptoglobin β (10 or 100 μg/mouse) was given once a day for 3 days starting at 24 hours after CLP surgery (N=25 mice/group). *:P<0.05 vs. control group.

FIG. 6A-6C: 6A. Schematics of haptoglobin (Hp) β and Hp β peptide (SEQ ID NO:1). 6B. BIAcore analysis revealed that Hpβ peptide (SEQ ID NO:1) binds HMGB1 (Kd≈17 μM). 6C. In primary mouse macrophages, addition of Hpβ peptide (SEQ ID NO:1) dose-dependently inhibited HMGB1-induced TNF release. Similar results were observed using RAW 264.7 or primary human macrophages. *: p<0.05 vs. HMGB1 alone. N=5.

FIG. 7: Haptoglobin peptide sequences tested. (Top to bottom, peptides 1-11, respectively; SEQ ID NOS. 4, 5, 6, 7, 1, 8, 9, 10, 11, 12 and 13, respectively)).

### DETAILED DESCRIPTION OF THE INVENTION

A method of treating sepsis in a subject is provided, comprising administering to the subject an amount of composition comprising a peptide having SEQ ID NO:1, but not comprising SEQ ID NO:2, effective to treat sepsis in a subject.

Also provided is a method of reducing the likelihood of mortality from sepsis in a subject having the sepsis, comprising administering to the subject an amount of composition comprising a peptide having SEQ ID NO:1, but not comprising SEQ ID NO:2, effective reduce the likelihood of mortality of a subject from sepsis.

Also provided is a method of inhibiting HMGB1-induced TNF release from a macrophage in a subject, comprising administering to the subject an amount of composition comprising a peptide having SEQ ID NO:1, but not comprising SEQ ID NO:2, effective to inhibit HMGB1-induced TNF release from a macrophage in a subject.

Also provided is a method of treating acetaminophen-induced liver damage in a subject, comprising administering to the subject an amount of composition comprising a peptide having SEQ ID NO:1, but not comprising SEQ ID NO:2, effective to treat acetaminophen-induced liver damage in a subject.

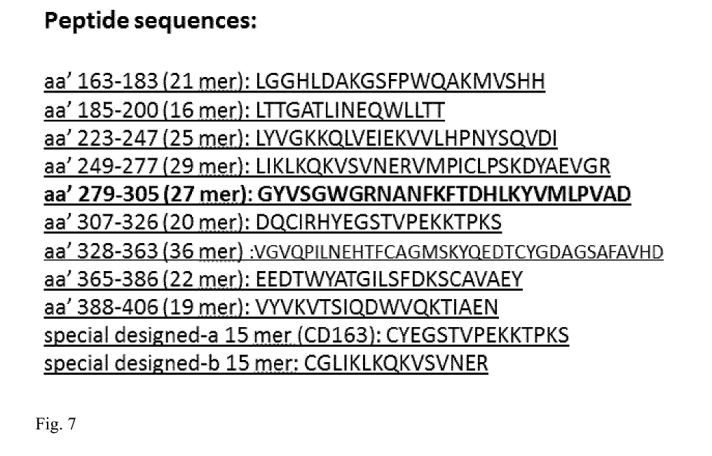

Peptide #5 (See, e.g., FIG. 7).
(SEQ ID NO: 1)
GYVSGWGRNANFKFTDHLKYVMLPVAD.
Peptide #4 (See, e.g., FIG. 7).
(SEQ ID NO: 7)
LIKLKQKVSVNERVMPICLPSKDYAEVGR.
Haptoglobin mature sequence (human):
(SEQ ID NO: 2)
VDSGNDVTDIADDGCPKPPEIAHGYVEHSVRYQCKNYYKLRTEGDGVYTL
NDKKQWINKAVGDKLPECEADDGCPKPPEIAHGYVEHSVRYQCKNYYKLR
TEGDGVYTLNNEKQWINKAVGDKLPECEAVCGKPKNPANPVQRILGGHLD
AKGSFPWQAKMVSHHNLTTGATLINEQWLLTTAKNLFLNHSENATAKDIA
PTLTLYVGKKQLVEIEKVVLHPNYSQVDIGLIKLKQKVSVNERVMPICLP
SKDYAEVGRVGYVSGWGRNANKFTDHLKYVMLPVADQDQCIRHYEGSTVP
EKKTPKSPVGVQPILNEHTFCAGMSKYQEDTCYGDAGSAFAVHDLEEDTW
YATGILSFDKSCAVAEYGVYVKVTSIQDWVQKTIAEN
Haptoglobin beta subunit (human):
(SEQ ID NO: 3)
ILGGHLDAKGSFPWQAKMVSHHNLTTGATLINEQWLLTTAKNLFLNHSEN
ATAKDIAPTLTLYVGKKQLVEIEKVVLHPNYSQVDIGLIKLKQKVSVNER
VMPICLPSKDYAEVGRVGYVSGWGRNANFKFTDHLKYVMLPVADQDQCIR
HYEGSTVPEKKTPKSPVGVQPILNEHTFCAGMSKYQEDTCYGDAGSAFAV
HDLEEDTWYATGILSFDKSCAVAEYGVYVKVTSIQDWVQKTIAEN

In an embodiment of the methods, the composition is administered intravenously. Alternative routes of administration embodied herein are auricular, buccal, conjunctival, cutaneous, subcutaneous, endocervical, endosinusial, endotracheal, enteral, epidural, via hemodialysis, interstitial, intrabdominal, intraamniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronary, intradermal, intradiscal, intraductal, intraepidermal, intraesophagus, intragastric, intravaginal, intragingival, intraileal, intraluminal, intralesional, intralymphatic, intramedullary, intrameningeal, intramuscular, intraocular, intraovarian, intraepicardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intraventricular, intravesical, intravitreal, laryngeal, nasal, nasogastric, ophthalmic, oral, oropharyngeal, parenteral, percutaneous, periarticular, peridural, rectal, inhalationally, retrobulbar, subarachnoid, subconjuctival, sublingual, submucosal, topically, transdermal, transmucosal, transplacental, transtracheal, ureteral, uretheral, and vaginal.

In an embodiment of the methods, the peptide is recombinantly produced.

In an embodiment of the methods, the peptide is fused to a molecule that increases plasma-half life of the peptide. In an embodiment of the methods, the peptide is fused to an XTEN molecule, a PEG molecule, or an albumin molecule.

In an embodiment, the peptide is administered as a fusion protein. In an embodiment, the peptide is fused to a portion of an immunoglobulin, e.g. a portion of an IgG or an IgM. In an embodiment, it as a portion of an IgG. The IgG portion of the fusion protein can be, e.g., any of an IgG1, IgG2, IgG2a, IgG2b, IgG3 or IgG4 or a portion thereof. In an embodiment, the portion is an Fc region. In an embodiment the fusion protein comprises a sequence identical to an Fc portion of a human IgG1, human IgG2, human IgG2a, human IgG2b, human IgG3 or human IgG4. In an embodiment the fusion protein comprises a sequence identical to an Fc portion of a human IgG1. The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine of the Fc region may be removed, for example, by recombinantly engineering the nucleic acid encoding the fusion protein. In an embodiment, the peptide is linked to the Fc domain through a linker. In an embodiment, it is linked via a peptide linker which permits flexibility. In an embodiment, the linker is rigid. In an embodiment the linker is cleavable. Non-limiting examples of flexible linkers within the scope of the invention are Gₙ, and GGGGS, and (GGGGS)ₙ where n=2, 3, 4 or 5. Non-limiting examples of rigid linkers within the scope of the invention are (EAAAK)ₙ, (XP)ₙ. Non-limiting examples of cleavable linkers within the scope of the invention include disulfide links and protease cleavable linkers. In a preferred embodiment, the linker is a peptide linker.

In an embodiment, the Fc domain has the same sequence or 95% or greater sequence similarity with a human IgG1 Fc domain. In an embodiment, the Fc domain has the same sequence or 95% or greater sequence similarity with a human IgG2 Fc domain. In an embodiment, the Fc domain has the same sequence or 95% or greater sequence similarity with a human IgG3 Fc domain. In an embodiment, the Fc domain has the same sequence or 95% or greater sequence similarity with a human IgG4 Fc domain. In an embodiment, the Fc domain is not mutated. In an embodiment, the Fc domain is mutated at the CH2-CH3 domain interface to increase the affinity of IgG for FcRn at acidic but not neutral pH (Dall'Acqua et al, 2006; Yeung et al, 2009).

In an embodiment, the fusion protein described herein is recombinantly produced. In an embodiment, the fusion protein is produced in a eukaryotic expression system. In an embodiment, the fusion protein produced in the eukaryotic expression system comprises glycosylation at a residue on the Fc portion corresponding to Asn297.

In an embodiment, the fusion protein is a homodimer. In an embodiment, the fusion protein is monomeric. In an embodiment, the fusion protein is polymeric.

In an embodiment of the methods, the peptide consists of L-amino acids. In an embodiment of the methods, the peptide comprises L-amino acids and D-amino acids. In an embodiment of the methods, the peptide consists of D-amino acids.

As used herein, “treating” sepsis means that one or more symptoms of the disease, such as inflammation, cytokine release, organ dysfunction, or other parameters by which the disease is characterized, are reduced, ameliorated, prevented, or placed in a state of retreat.

As used herein, “treating” acetaminophen-induced liver damage means that one or more symptoms of the disease or other parameters by which the disease is characterized, are reduced, ameliorated, or prevented.

Also provided is a composition comprising a peptide having SEQ ID NO:1, but not comprising SEQ ID NO:2, wherein the peptide is fused to a molecule that increases plasma-half life of the peptide.

In an embodiment of the composition, the peptide is fused to an XTEN molecule, a PEG molecule, or an albumin molecule. For XTEN protein see, e.g. Podust et al., Journal of Controlled Release, Volume 240, 28 Oct. 2016, Pages 52-66; also see Schellenberger et al., Nat Biotechnol. 2009 December; 27(12):1186-90, each hereby incorporated by reference. In an embodiment of the composition, the peptide is modified to be an azatide derivative of a peptide.

In an embodiment of the composition, the peptide consists of L-amino acids.

In an embodiment of the composition, the peptide comprises L-amino acids and D-amino acids.

In an embodiment of the composition, the peptide consists of D-amino acids.

In an embodiment of the composition, the composition comprises a pharmaceutically acceptable carrier.

The invention provides, a composition comprising an isolated peptide which is not a contiguous part of the native haptoglobin sequence, wherein the peptide is any one of SEQ ID NOS: 1 or 4-13. In an embodiment, the peptide is conjugated to a molecule that increases plasma-half life of the peptide. In an embodiment of the composition, the peptide is fused to an XTEN molecule, a PEG molecule, or an albumin molecule. In an embodiment of the composition, the peptide is modified to be an azatide derivative of a peptide.

“Carrier”: The term “carrier” is used in accordance with its art-understood meaning, to refer to a material that is included in a pharmaceutical composition but does not abrogate the biological activity of pharmaceutically active agent(s) that are also included within the composition. Typically, carriers have very low toxicity to the animal to which such compositions are to be administered. In some embodiments, carriers are inert. In some embodiments, carriers are affirmatively beneficial. In some embodiments, the term “carrier” when used in the pharmaceutical context (e.g., pharmaceutically acceptable carrier) means that an agent is present in a composition but does not abrogate the biological activity of another agent(s) present in a composition, for example the peptide of the composition.

“Pharmaceutically acceptable”: The term “pharmaceutically acceptable” as used herein applied to carriers refers to those carriers which are, within the scope of medical judgment, suitable for use in contact with the tissues of humans and lower animals without undue toxicity, irritation, allergic response, and the like, commensurate with a reasonable benefit/risk ratio, and effective for their intended use.

The compositions of the inventions can comprise one or more additional components which facilitate use of the composition in treating sepsis or liver damage, or which enhance storage properties of the composition. For example, pH adjusting agent, and preservative(s).

“pH adjusting agent”: As used herein, the term “pH adjusting agent” as used herein is an agent that imparts suitable pH characteristics to compositions provided herein, (e.g., a substantially neutral pH, e.g. pH 7.35), the pH of which depends on the specific utilization of the composition. Suitable pH adjusting agents include, for example, but are not limited to, one or more adipic acids, buffers, citric acids, calcium hydroxides, glycines, magnesium aluminometasilicates, or combinations thereof.

“Preservative”: As used herein, the term “preservative” has its art-understood meaning and refers to an agent that protects against undesirable chemical modifications of one or more components in a composition (e.g., protection against an undesirable chemical modification of an active ingredient). Suitable preservatives for use in the compositions of the present invention include, but are not limited to, one or more alkanols, disodium EDTA, EDTA salts, EDTA fatty acid conjugates, isothioazolinone, parabens such as methylparaben and propylparaben, polypropylene glycols, sorbates, urea derivatives such as diazolindinyl urea, or combinations thereof.

A method of treating sepsis in a subject is provided, comprising administering to the subject an amount of composition comprising a peptide having SEQ ID NO:1, but not comprising SEQ ID NO:2, effective to treat sepsis in a subject.

Also provided is a method of reducing the likelihood of mortality from sepsis in a subject having the sepsis, comprising administering to the subject an amount of composition comprising a peptide having SEQ ID NO:7, but not comprising SEQ ID NO:2, effective reduce the likelihood of mortality of a subject from sepsis.

Also provided is a method of inhibiting HMGB1-induced TNF release from a macrophage in a subject, comprising administering to the subject an amount of composition comprising a peptide having SEQ ID NO:7, but not comprising SEQ ID NO:2, effective to inhibit HMGB1-induced TNF release from a macrophage in a subject.

Also provided is a method of treating acetaminophen-induced liver damage in a subject, comprising administering to the subject an amount of composition comprising a peptide having SEQ ID NO:7, but not comprising SEQ ID NO:2, effective to treat acetaminophen-induced liver damage in a subject.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### Experimental Details

During a search for host molecules that contribute to the pathogenesis of severe sepsis, the inventors discovered that free hemoglobin significantly increases LPS-mediated toxicity. It was reasoned that removal of free hemoglobin would be protective against tissue damage and lethality in sepsis. To study this hypothesis, an extra-corporeal haptoglobin affinity chromatography method was developed to remove extracellular hemoglobin in rodents with sepsis induced by cecal ligation and puncture surgery. Surprisingly, it was observed that haptoglobin-affinity chromatography extracted HMGB1 from the blood of septic rats. Subsequent study of underlying mechanisms indicates that haptoglobin forms a complex with HMGB1 to stimulate macrophage HO-1 production through a CD163-dependent pathway. Moreover, haptoglobin β subunit alone is sufficient to recapitulate the HMGB1-binding effects of full-length haptoglobin (SEQ ID NO:2). Thus, herein is revealed that the structural basis of HMGB1-binding of haptoglobin is located at its 13 subunit, and specifically to a 26-mer peptide (SEQ ID NO:1) in the 13 subunit (SEQ ID NO:3).

Secreted by activated cells or passively released by damaged cells, extracellular HMGB1 is a prototypical damage-associated molecular pattern (DAMP) inflammatory mediator. During the course of developing extracorporeal approaches to treating injury and infection, it was discovered that haptoglobin, the acute phase protein that binds extracellular hemoglobin and targets cellular uptake through CD163, also binds HMGB1. Hapotglobin-HMGB1 complexes elicit the production of anti-inflammatory enzymes (heme oxygenease-1) and cytokines (e.g., IL-10) in wild type, but not in CD163-deficient macrophages. Genetic disruption of haptoglobin or CD163 expression significantly enhances mortality rates in standardized models of intra-abdominal sepsis (CLP) in mice. Administration of haptoglobin to wild type and to haptoglobin-gene deficient animals confers significant protection.

Haptoglobin is a complex protein consisting of α and β subunits (Yueh S C et al. J Chromatography, 2007). Structural functional analysis revealed that haptoglobin β alone recapitulate the HMGB1-binding effects of the full-length protein. Haptoglobin β subunit binds HMGB1 with similar high affinity as compared to the full length protein. Administration of haptoglobin β subunit protects against lethality in mice with CLP-induced sepsis (CLP survival=85% in haptoglobin β-treated versus 50% in vehicle-treated group; n=22 mice per group, P<0.05) and in experimental acetaminophen-induced liver injury (Survival in haptoglobin β treated group=80% versus 35% in vehicle control group, P<0.05, n=20 mice per group). Screening of a peptide library of the haptoglobin β identified a critical region (26 amino acids, residues #278-305 in the B subunit) that retains the ability to inhibit HMGB1-induced TNF release from cultured macrophages. Taken together, these findings reveal a novel mechanism for haptoglobin modulation of the inflammatory action of HMGB1, with significant implications for developing experimental strategies targeting HMGB 1-dependent inflammatory diseases.

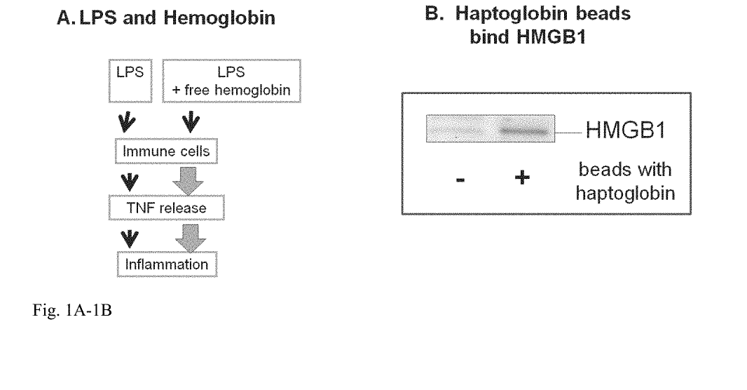

Free hemoglobin enhances endotoxin toxicity: As shown in FIG. 1, Free hemoglobin is ubiquitous and enhances LPS-mediated inflammation. Initially, a treatment method was developed using haptoglobin-beads to remove free hemoglobin from septic rats. Unexpectedly, HMGB1 was pulled out using haptoglobin linked to sepharose beads in an extracorporeal circulatory device, besides hemoglobin.

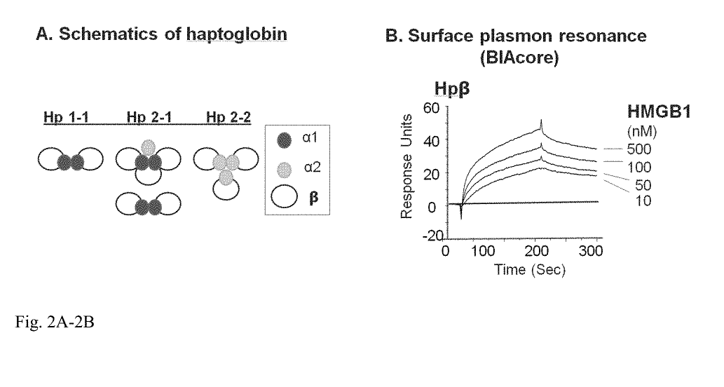

Haptoglobin (Hp) β binds to HMGB1: Human haptoglobin has three phenotypes and contains α and β subunits. Similarly to full length haptoglobin (Yang, JCI Insight, 2016), haptoglobin β binds to HMGB1 with high affinity (Kd=29 nM) and in a concentration-dependent manner using surface plasmon resonance analysis. Data shown in FIG. 2 demonstrating this is representative of 3 separate experiments.

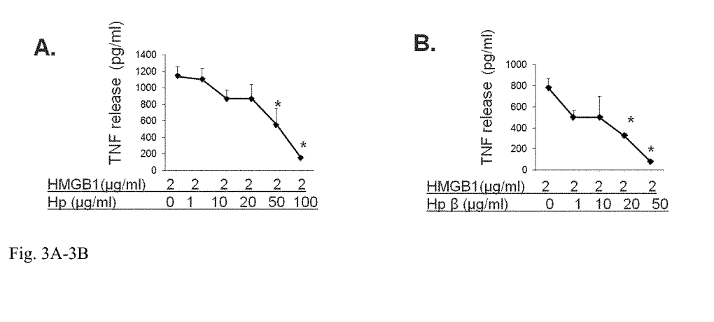

Haptoglobin and haptoglobin (3, inhibits HMGB1-induced TNF release from macrophages: As shown in FIG. 3, HMGB1 stimulated TNF release in murine macrophage-like RAW 264.7 cells. FIG. 3A shows the addition of haptoglobin produced a dose-dependent attenuation of the HMGB1-induced TNF release (up to 90% inhibition at 100 ug/ml), and FIG. 3B shows haptoglobin β is as effective as haptoglobin in inhibiting HMGB1-induced TNF release.

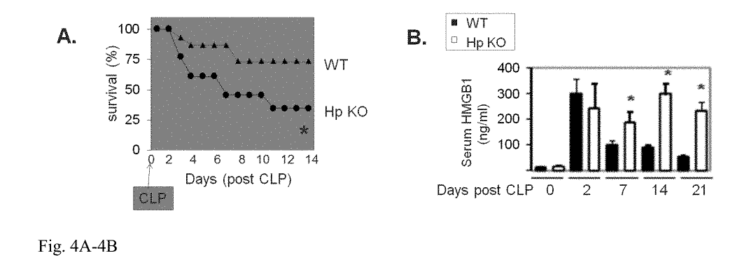

Haptoglobin knockout (KO) mice have higher sepsis mortality and elevated serum HMGB1 levels: To elucidate the links between haptoglobin, HMGB1 and sepsis, mice were studied with Hp knockout (KO). Hp KO mice, following CLP, suffered a doubling of mortality compared to wild type mice. In Hp KO mice after CLP, serum HMGB1 levels were persistently higher for at least 21 days compared to wild type mice, suggesting HMGB1 may play a role in increased mortality in Hp KO mice. (See FIG. 4A-B).

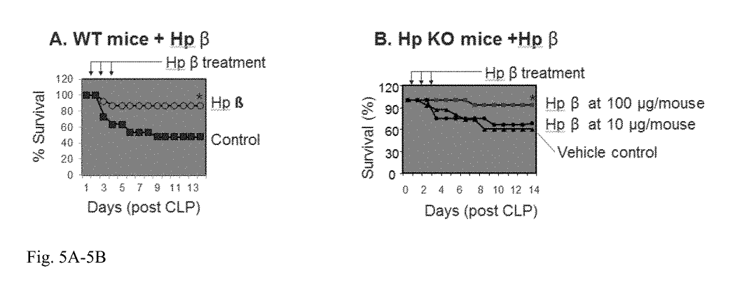

Haptoglobin β protects against death from sepsis: Wild type mice subjected to cecal ligation and puncture (CLP) who received injections of haptoglobin β (200 μg/mouse injected IP) were twice as likely to survive as compared to vehicle-treated (control) group (*: P<0.05). N=16 mice/group. Haptoglobin KO mice were rescued from death by administration of haptoglobin β at doses as low as 100 μg per animal per day. Recombinant haptoglobin β (10 or 100 μg/mouse) was given once a day for 3 days starting at 24 hours after CLP surgery (N=25 mice/group). (See FIG. 5A-5B).

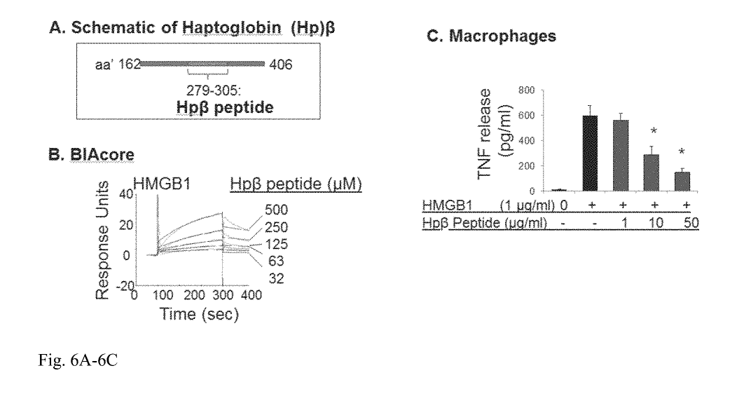

Haptoglobin β peptide retains the activity to bind and inhibit HMGB1 toxicity. Schematics of haptoglobin (Hp) β and Hp β peptide are shown in FIG. 6A. BIAcore analysis revealed that Hpβ peptide binds HMGB1 (Kd≈17 μM) (FIG. 6B). In primary mouse macrophages, addition of Hpβ peptide dose-dependently inhibited HMGB1-induced TNF release. Similar results were observed using RAW 264.7 or primary human macrophages. (See FIG. 6C).

Acetaminophen-induced liver injury: Administration of haptoglobin β subunit protects against lethality in mice with experimental acetaminophen-induced liver injury (survival in haptoglobin β treated group=80% versus 35% in vehicle control group, P<0.05, n=20 mice per group).

What is claimed is:

1. A method of treating sepsis in a subject, comprising administering to the subject an amount of composition comprising a peptide having SEQ ID NO:1 but not comprising SEQ ID NO:2, effective to treat sepsis in a subject.

2. A method of reducing the likelihood of mortality from sepsis in a subject having the sepsis, comprising administering to the subject an amount of composition comprising a peptide having SEQ ID NO:1 but not comprising SEQ ID NO:2, effective reduce the likelihood of mortality of a subject from sepsis.

3. A method of inhibiting HMGB1-induced TNF release from a macrophage in a subject, comprising administering to the subject an amount of composition comprising a peptide having SEQ ID NO:1 but not comprising SEQ ID NO:2, effective to inhibit HMGB1-induced TNF release from a macrophage in a subject.

4. A method of treating acetaminophen-induced liver damage in a subject, comprising administering to the subject an amount of composition comprising a peptide having SEQ ID NO:1 but not comprising SEQ ID NO:2, effective to treat acetaminophen-induced liver damage in a subject.

5. The method of claim 1, wherein the composition is administered intravenously.

6. The method of claim 1, wherein the peptide is recombinantly produced.

7. The method of claim 1, wherein the peptide is fused to a molecule that increases plasma-half life of the peptide.

8. The method of claim 1, wherein the peptide is fused to an XTEN molecule, a PEG molecule, or an albumin molecule.

9. The method of claim 1, wherein the peptide consists of L-amino acids.

10. The method of claim 1, wherein the peptide comprises L-amino acids and D-amino acids.

11. The method of claim 1, wherein the peptide consists of D-amino acids.

12. The method of claim 1, wherein the composition comprises a pharmaceutically acceptable carrier.

13. A composition comprising a peptide having SEQ ID NO:1 but not comprising SEQ ID NO:2, wherein the peptide is fused to a molecule that increases plasma-half life of the peptide.

14. The composition of claim 13, wherein the peptide is fused to an XTEN molecule, a PEG molecule, or an albumin molecule.

15. The composition of claim 13, wherein the peptide consists of L-amino acids.

16. The composition of claim 13, wherein the peptide comprises L-amino acids and D-amino acids.

17. The composition of claim 13, wherein the peptide consists of D-amino acids.

18. The composition of claim 13, wherein the composition comprises a pharmaceutically acceptable carrier.
